(19) United States
(12) Patent Application Publication
(10) Pub. No.: US 2023/0038541 A1
(43) Pub. Date: Feb. 9, 2023

(54) BRAIN STIMULATION SIMULATION SYSTEM AND METHOD ACCORDING TO PRESET GUIDE SYSTEM USING ANONYMIZED DATA-BASED EXTERNAL SERVER

(71) Applicant: NEUROPHET Inc., Seoul (KR)

(72) Inventor: Dong Hyeon KIM, Seoul (KR)

(21) Appl. No.: 17/676,758

(22) Filed: Feb. 21, 2022

(30) Foreign Application Priority Data
Jul. 7, 2021 (KR) 10-2021-0089052

(51) Int. Cl. G06F 30/23; G06T 17/20; G06T 7/10

(52) U.S. Cl. CPC G06F 30/23; G06T 17/20; G06T 7/10; G06T 2210/41; G06T 2207/10088

(57) ABSTRACT

A brain stimulation simulation system and method according to a preset guide system using an anonymized data-based external server are provided. According to various embodiments of the present invention, provided is a brain stimulation simulation method according to a preset guide system using an external server, the method performed by a computing device, the method including: a first server generating a global matrix for performing brain stimulation simulation on a plurality of objects by using a plurality of brain models for each of the plurality of objects; and a second server being provided with the generated global matrix from the first server and performing the brain stimulation simulation on the plurality of objects by using the provided global matrix.

### CROSS-REFERENCE TO RELATED APPLICATION

This application is based on and claims priority under 35 U.S.C. 119 to Korean Patent Application No. 10-2021- 0089052, filed on Jul. 7, 2021, in the Korean Intellectual Property Office, the disclosure of which is herein incorporated by reference in its entirety.

### TECHNICAL FIELD

Various embodiments of the present invention relate to a brain stimulation simulation system and method according to a preset guide system using an anonymized data-based external server.

### BACKGROUND ART

The brain is an internal organ of a human head and is the highest central organ of a nervous system. The brain is divided into cerebrum, cerebellum, midbrain, pons, and medulla oblongata. In addition, the brain generates a brain wave which is a signal obtained by measuring a total sum of neuronal activity levels in an epidermis of the brain.

As a method for measuring a state of the brain, there are an EEG (electroencephalogram) test, which measures and examines the brain waves received from electrodes by attaching pads with electrodes to a scalp, a CT scan which examines the brain by taking tomography from various angles using radiation or ultrasound, an MRI scan which images the brain by magnetic resonance, and the like.

Various concepts are known in the field of neural stimulation of brain structures, and brain stimulation which stimulates the brain to achieve a predetermined purpose is largely classified into invasive brain stimulation and non-invasive brain stimulation.

The invasive brain stimulation is a method in which electrodes are inserted into the brain through surgery and electrical signals are applied, and the non-invasive brain stimulation is a method in which a predetermined effect is achieved by stimulating the brain without inserting the electrodes inside a skull.

Specific brain stimulation includes deep electric stimulation, transcranial magnetic stimulation (TMS), transcranial electric stimulation (TES), transcranial direct current stimulation (tDCS), and transcranial random noise stimulation (tRNS).

Among these brain stimulations, a brain electric stimulation technology using the transcranial direct current stimulation (tDCS) is one of the relatively simple non-invasive brain stimulations that is known to be able to improve cognitive abilities or to be effective in treating various cranial nerve diseases such as depression, attention deficit hyperactivity disorder (ADHD), epilepsy, dementia, and sleep disorders, and thus, the brain stimulations are actively studied.

In the method for stimulating the brain by using the transcranial direct current stimulation (tDCS) device, an anode and a cathode are connected to a transcranial direct current stimulation (tDCS) device that generates a direct current, and when a current is injected into the anode, the current passes through the cerebrum and comes back into the cathode.

In this case, the current flows from the anode to the cathode to stimulate the cerebrum, and it may be necessary to change the direction of the electric stimulation according to the treatment method. In the related art, in order to accurately stimulate a preset target point in the brain according to the transcranial direct current stimulation method, a process of performing brain stimulation simulation by using the brain model of the user needs to be performed in advance, but in general, the performance of the computing device provided in the medical institution such as hospitals is not good. Therefore, there is a problem in that a lot of time is taken in a simulation process and the simulation for a plurality of users cannot be performed at the same time.

In addition, when the brain stimulation simulation for the plurality of users using an external computing device with better performance is to be performed, medical data including information about the plurality of users need to be exported to the outside, and thus, legal sanctions (for example, a personal information protection act or the like) are applied. Therefore, there is a problem in that, in order to prevent this sanction, a process of de-identification of a huge amount of medical data needs to be performed.

### SUMMARY OF INVENTION

### Technical Problem

An object of the present invention is to overcome the problem of the related art and, thus, is to provide a brain stimulation simulation system and method according to a preset guide system using an anonymized data-based external server capable of preventing legal sanctions by guaranteeing anonymity for a large number of users when transmitting medical data to the outside by generating a global matrix not including information about the users and transmitting the global matrix to an external server in order to perform brain stimulation simulation for a large number of users to an external server.

And another object of the present invention is to provide a brain stimulation simulation system and method according to a preset guide system using an anonymized data-based external server capable of processing the simulation for a plurality of the users faster and more accurately through an external computing device with better performance by performing the global matrix-based brain stimulation simulation through the external server and being provided with a result of the performed brain stimulation simulation.

The objects of the present invention are not limited to the object mentioned above, and other objects not mentioned will be clearly understood from the following description by the ordinarily skilled in the art.

### Solution to Problem

According to one embodiment of the present invention, there is provided a brain stimulation simulation method according to a preset guide system using an external server, the method performed by a computing device, the method including: a first server generating a global matrix for performing brain stimulation simulation on a plurality of objects by using a plurality of brain models for each of the plurality of objects; and a second server being provided with the generated global matrix from the first server and performing the brain stimulation simulation on the plurality of objects by using the provided global matrix.

In various embodiments, the generating the global matrix may include: acquiring an MRI image of the plurality of objects; segmenting the acquired MRI image into a plurality of areas; generating a three-dimensional brain image by using the MRI image segmented into the plurality of areas; generating a three-dimensional brain map configured with a plurality of meshes based on properties of each of the plurality of areas included in the generated three-dimensional brain image; and generating the global matrix by using the generated three-dimensional brain map.

In various embodiments, the generating the global matrix by using the generated three-dimensional brain map may include: deriving a mathematical formula for performing the brain stimulation simulation; grouping a plurality of nodes included in the generated three-dimensional brain map into a plurality of groups and generating a unit matrix for each of the plurality of groups by using the derived mathematical formula; and generating one global matrix by combining the generated unit matrix.

In various embodiments, the deriving the mathematical formula may include deriving the mathematical formula for performing the brain stimulation simulation, a form of the derived mathematical formula being determined according to a purpose of performing the brain stimulation simulation, wherein the purpose includes at least one among time-series current prediction, constant current and low-frequency current prediction, and vibration prediction for ultrasound stimulation.

In various embodiments, each of the plurality of groups may include four nodes forming a tetrahedral shape, and the generating the unit matrix for each of the plurality of groups may include generating a stiffness matrix for the four nodes forming the tetrahedral shape by using a finite element method (FEM),

In various embodiments, the segmenting into the plurality of area may include allocating a physical property for each of the plurality of areas to each of the plurality of areas generated by segmenting the acquired MRI image, a type of the physical property allocated to each of the plurality of areas being determined according to a type of brain stimulation to be simulated.

In various embodiments, the generating the global matrix may further include setting a brain stimulation condition for performing the brain stimulation simulation, the set brain stimulation condition including at least one of a plurality of stimulation positions according to the preset guide system, a number of electrodes attachable to the plurality of stimulation positions, and an intensity of brain stimulation.

In various embodiments, the method may further include the first server being provided with a result of brain stimulation simulation for the plurality of objects from the second server and outputting a combination of the provided result of the brain stimulation simulation and the generated three-dimensional brain map.

In various embodiments, the performing the brain stimulation simulation may include: deriving a linear equation for the provided global matrix by using the provided global matrix; and calculating a solution of the derived linear equation as the result of the brain stimulation simulation.

According to another embodiment of the present invention, there is provided a brain stimulation simulation system according to a preset guide system using an external server, including: a first server generating a global matrix for performing brain stimulation simulation on a plurality of objects by using a plurality of brain models for each of the plurality of objects; and a second server being provided with the generated global matrix from the first server and performing the brain stimulation simulation on the plurality of objects by using the provided global matrix.

Other specific details of the present invention are included in the detailed description and the drawings.

### Advantageous Effects of Invention

According to various embodiments of the present invention, it has advantages of capable of preventing legal sanctions by guaranteeing anonymity for a large number of users when transmitting medical data to the outside by generating a global matrix not including information about the users and transmitting the global matrix to an external server in order to perform brain stimulation simulation for a large number of users to an external server and capable of processing the simulation for a plurality of the users faster and more accurately through an external computing device with better performance by performing the global matrix-based brain stimulation simulation through the external server and being provided with a result of the performed brain stimulation simulation.

Effects of the present invention are not limited to the effects mentioned above, and other effects not mentioned will be clearly understood from the following description by those skilled in the art.

### BRIEF DESCRIPTION OF DRAWINGS

FIG. 1 is a diagram illustrating a brain stimulation simulation system according to a preset guide system using an external server according to an embodiment of the present invention.

FIG. 2 is a hardware configuration diagram illustrating a first server of the brain stimulation simulation system according to the preset guide system using the external server according to various embodiments.

FIG. 3 is a flowchart illustrating the brain stimulation simulation method according to the preset guide system using the external server according to another embodiment of the present invention.

FIG. 4 is a flowchart illustrating a method of generating a three-dimensional brain map using a brain image of an object in various embodiments.

FIG. 5 is an exemplary diagram illustrating an MRI image of the brain of the object and a result of segmenting the MRI image in various embodiments.

FIG. 6 is a diagram illustrating a process of rejecting noise in an MRI image segmented into a plurality of areas by performing noise rejection based on a connected component in various embodiments.

FIG. 7 is a diagram illustrating a process of generating a three-dimensional brain image by performing hole rejection processing on the MRI image segmented into the plurality of areas in various embodiments;

FIG. 8 is a flowchart illustrating a method of generating a global matrix in various embodiments.

FIG. 9 is an exemplary diagram illustrating a plurality of stimulation positions according to the preset guide system applicable to various embodiments.

FIG. 10 is a flowchart illustrating a method for performing brain stimulation simulation by filtering the stimulation positions in various embodiments.

FIG. 11 is an exemplary diagram illustrating a form of filtering at least one stimulation position by setting a filtering target area in various embodiments.

FIG. 12 is an exemplary diagram illustrating a user interface (UI) provided by a first server in various embodiments.

FIG. 13 is an exemplary diagram illustrating a three-dimensional brain map in which results of the brain stimulation simulation are reflected in various embodiments.

### DESCRIPTION OF EMBODIMENTS

Advantages and features of the present invention and methods of achieving the advantages and features will become apparent with reference to embodiments described below in detail in association with the accompanying drawings. However, the present invention is not limited to the embodiments disclosed below, but the present invention can be implemented in various different forms, only the embodiments allow the disclosure of the present invention to be complete, the present invention is provided in order for the ordinarily skilled in the art to which the present invention belongs to fully understand the scope of the present invention, and the present invention is only defined by the scope of the claims.

The terminology used herein is for the purpose of describing the embodiments and is not intended to limit the present invention. In this specification, a singular form also includes a plural form unless a phrase specifically states otherwise. As used in this specification, “comprises” and/or “comprising” do not exclude the presence or addition of one or more other components in addition to the stated components. Throughout the specification, the same or similar reference numerals refer to the same or similar elements, and “and/or” includes each and all combination of one or more of the stated elements. Although “first”, “second”, and the like are used to describe various elements, of course, these elements are not limited by these terms. These terms are only used to distinguish one component from other components. Accordingly, it goes without saying that a first component mentioned below may be a second component within the spirit of the present invention.

Unless otherwise defined, all terms (including technical and scientific terms) used herein may be used with the meaning commonly understood by the ordinarily skilled in the art to which the present invention belongs. In addition, terms defined in a commonly used dictionary are not to be interpreted ideally or excessively unless specifically defined explicitly.

As used in this specification, the term “unit” or “module” refers to a software component or a hardware component such as FPGA or ASIC, and the “unit” or “module” performs a certain role. However, the “unit” or “module” is not meant to be limited to software or hardware. The “unit” or “module” may be configured to reside on an addressable storage medium or may be configured to reproduce one or more processors. Accordingly, as an example, the “unit” or “module” includes components such as software components, object-oriented software components, class components and task components, processes, functions, attributes, procedures, subroutines, segments of program codes, drivers, firmware, microcode, circuits, data, database, data structures, tables, arrays, and variables. The components and functions provided within the “unit” or “module” may be combined into a smaller number of components and “units” or “modules” or may be further separated in to additional components and “units” or “modules”.

Spatially relative terms “below”, “beneath”, “lower”, “above”, “upper”, and the like can be used to easily describe the relationship between a certain component and other components. Spatially relative terms should be understood as terms that include different directions of components during use or operation in addition to the directions illustrated in the drawings. For example, in a case where a component illustrated in the drawings is turned over, a component described as “below” or “beneath” of the other component may be placed “above” of the other component. Accordingly, the exemplary term “below” may include both directions below and above. Components may also be oriented in other orientations, and thus, spatially relative terms may be interpreted according to orientation.

In this specification, a computer denotes all types of hardware devices including at least one processor and may be understood as collectively including software configurations operating in a corresponding hardware device according to embodiments. For example, a computer may be understood as meaning including all of a smartphone, a tablet PC, a desktop, a notebook, and a user client and an application running in each device, but the present invention is not limited thereto.

Hereinafter, an embodiment of the present invention will be described in detail with reference to the accompanying drawings.

Each step described in this specification is described as being performed by a computer, but the subject of each step is not limited thereto, and at least a portion of each step may be performed in different devices according to embodiments.

Analysis of the medical data is generally performed simply by using a computing device (or computer) within a medical institution such as a hospital. Due to the development in medical technology, the analysis of the medical data is highly advanced, and thus, the need for services of analyzing the medical data by using computing devices outside the medical institution, such as cloud services, is increasing. Therefore, various medical data analysis services are emerging.

As in a cloud service or the like, when the medical data is to be analyzed by using a computing device outside the medical institution, a process of transmitting the medical data stored in the computing device within the medical institution to the computing device outside the medical period is needed. However, since the medical data contain various information of patients (for example, personal information of the patients), when there is no patient's permission or the medical data are leaked outside the hospital or outside the country without de-identification, there is an issue in that legal sanctions under a personal information protection act or the like are applied.

Therefore, in order to analyze medical data through computing devices outside the medical institution, a de-identification process for personal information should be performed, such as removing identifiers (face contours, patient numbers) that can identify patients from medical images and removing patient identifiers (e.g., patient numbers, names, genders, and information that can infer that information) from genetic information or medical records.

More specifically, in the case of medical image data (for example a brain MRI image, a head CT image, an abdominal CT image, a three-dimensional ultrasound image, or the like.), software of deleting or masking identifiers such as patient number and name on the image, deleting identifiers in metadata such as a DICOM header, or the like, or deleting a surface boundary of body in an image information needs to be applied in accordance with a guideline. Therefore, there is a problem in that, in order to analyze the medical data through an external computing device, the medical data need to be corrected more complicatedly through a large number of processes and, thus, a lot of time and manpower are taken.

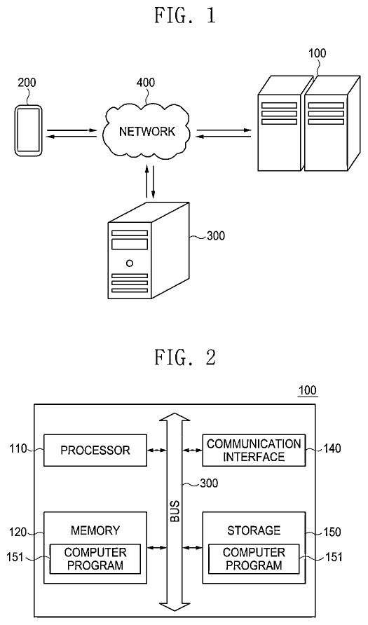

In order to overcome these problems, a brain stimulation simulation system and method according to a preset guide system using an external server according to various embodiments of the present invention is allowed to be capable of performing a large amount of computational processes (for example, simulation calculations for electrical brain stimulation, simulation calculations for ultrasound stimulation, calculations for source localization of EEG/MEG, or the like) required when performing the simulation for treatment design and analysis for a large number of patients through an external computing device in a state where the anonymity of a large number of patients is maintained. Hereinafter, the present invention will be described with reference to FIGS. 1 to 10.

FIG. 1 is a diagram illustrating a brain stimulation simulation system according to a preset guide system using an external server according to an embodiment of the present invention.

Referring to FIG. 1, the brain stimulation simulation system according to the preset guide system using the external server according to the embodiment of the present invention may include a first server 100, a second server 200, and a user terminal 300.

Herein, the brain stimulation simulation system according to the preset guide system using the external server illustrated in FIG. 1 is according to one embodiment. Components thereof are not limited to the embodiment illustrated in FIG. 1, and may be add or changed as necessary.

As an example, in some cases, the brain stimulation simulation system according to the preset guide system using the external server according to various embodiments of the present invention may include two or more first servers 100. In one second server 200, the brain stimulation simulation may be performed by using a global matrix generated by each of two or more first servers 100.

As another example, in some cases, the brain stimulation simulation system according to the preset guide system using the external server according to various embodiments of the present invention may include two or more second servers 200. The two or more second servers 200 may perform the brain stimulation simulation simultaneously or by segmenting global matrices generated by the first server 100 or may perform the brain stimulation simulation by respectively using each of the two or more global matrices generated by the first server 100.

In one embodiment, the first server 100 may generate the global matrix to perform the brain stimulation simulation on a plurality of objects through the second server 200 described later.

Herein, the global matrix may denote in form of a matrix not including personal information about multiple objects (patients who desire to simulate brain stimulation) in accordance with the Personal Information Protection Act and including only various types of information necessary for the brain stimulation simulation (for example, geometric structures of brain models of multiple objects, mathematical formulas, physical properties, or the like. necessary to perform the simulation), but the global matrix is not limited thereto.

In various embodiments, the first server 100 may selectively perform an operation of directly performing the brain stimulation simulation on the object and an operation of generating the global matrix for the brain stimulation simulation on the object.

For example, the first server 100 is a computing device provided in a medical institution such as a hospital, that is, a computing device with relatively low computing performance. When the number of objects for which the brain stimulation is to be simulated is less than or equal to a preset number, that is, when the brain stimulation simulation is to be performed on a small number of objects, the first server 100 may perform the brain stimulation simulation on a small number of objects by itself.

On the other hand, when the number of objects for which the brain stimulation is to be simulated is more than or equal to a preset number, that is, when the brain stimulation simulation is to be performed on a large number of objects, the first server 100 may generate the global matrix and provide the global matrix to the second server 200 so that the brain stimulation simulation can be performed on a large number of objects through the external second server 200.

In one embodiment, the second server 200 may be connected to the first server 100 through the network 400, may receive the global matrix from the first server 100, and may perform the brain stimulation simulation on the plurality of objects by using the provided global matrix.

In addition, the second server 200 may provide the result of performing the brain stimulation simulation on the plurality of objects by using the global matrix to the first server 100 through the network 400.

Herein, the second server 200 may be an external server which is separately provided outside a medical institution such as a hospital and has a relatively high-performance specification in comparison to the first server 100 so as to be capable of processing a process (for example, a process of simultaneously performing the brain stimulation simulation on a large number of objects) that is difficult to process in the first server 100, but the second server is not limited thereto.

In various embodiments, the first server 100 may be connected to the user terminal 300 through the network 400 and may perform the brain stimulation simulation on a specific object in response to a brain stimulation simulation request input through the user terminal 300 or may generate the global matrix for the brain stimulation simulation.

In addition, the first server 100 may provide the user terminal 300 with the result of performing the brain stimulation simulation on the plurality of objects in response to a brain stimulation simulation request input through the user terminal 300.

Herein, the user terminal 300 is a wireless communication device that guarantees portability and mobility and includes all types of handheld-based wireless communication device such as a navigation, a personal communication system (PCS), a global system for mobile communications (GSM), a personal digital cellular (PDC), a personal handyphone system (PHS), a personal digital assistant (PDA), an international mobile telecommunication (IMT)-2000, a code division multiple access (CDMA)-2000, a W-code division multiple access (W-CDMA), a wireless broadband internet (Wibro) terminal, a smartphone, a smart pad, and a tablet PC, but the user terminal is not limited thereto.

In addition, herein, the network 400 may have a connection structure capable of exchanging information between respective nodes such as a plurality of terminals and servers. For example, the network 400 includes a local area network (LAN), a wide area network (WAN), the Internet (WWW: World Wide Web), a wired/wireless data communication network, a telephone network, a wired/wireless television communication network, or the like.

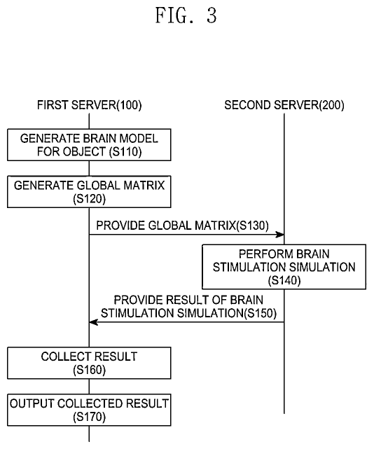

In addition, herein, the wireless data communication network includes 3G, 4G, 5G, a third generation partnership project (3GPP), a fifth generation partnership project (5GPP), a long term evolution (LTE), a world interoperability for microwave access (WIMAX), a Wi-Fi, the Internet, a local area network (LAN), a wireless local area network (Wireless LAN), a wide area network (WAN), a personal area network (PAN), a radio frequency (RF), a Bluetooth network, a near-field communication (NFC) network, a satellite broadcasting network, an analog broadcasting network, a digital multimedia broadcasting (DMB) network, or the like, but the wireless data communication network is not limited thereto. Hereinafter, a hardware configuration of the first server 100 performing the brain stimulation simulation method according to the preset guide system using the external server will be described with reference to FIG. 2.

FIG. 2 is a hardware configuration diagram illustrating the first server of the brain stimulation simulation system according to the preset guide system using the external server in the various embodiments.

Referring to FIG. 2, in various embodiments, the first server 100 may include one or more processor 110, a memory 120 on which a computer program 151 performed by the processor 110 is loaded, a bus 130, and a storage 150 storing a communication interface 140 and the computer program 151. Herein, only components related to the embodiment of the present invention are illustrated in FIG. 2. Accordingly, a person skilled in the art to which the present invention belongs can know that other general-purpose components other than the components illustrated in FIG. 2 may be further included. In addition, although the hardware configuration of the first server 100 is described below with reference to FIG. 2, the hardware configuration is not limited thereto, and the second server 200 may also include the same hardware configuration as the first server 100.

The processor 110 controls the overall operations of each configuration of the first server 100. The processor 110 may be configured to include a central processing unit (CPU), a micro process or unit (MPU), a micro controller unit (MCU), a graphic processing unit (GPU), or any type of processors well known in the art of the present invention.

In addition, the processor 110 may perform an operation for at least one application or program for executing the method according to embodiments of the present invention, and the first server 100 may include the one or more processors.

In various embodiments, the processor 110 may further include a random access memory (RAM, not illustrated) and a read only memory (ROM, not illustrated) temporarily and/or permanently storing signals (or data) processed inside the processor 110. In addition, the processor 110 may be implemented in a form of a system on chip (SoC) including at least one among a graphic processing unit, a RAM, and a ROM.

The memory 120 stores various data, commands and/or information. The computer program 151 from the storage 150 in order to perform methods/operations according to various embodiments of the present invention is loaded on the memory 120. When the computer program 151 is loaded on the memory 120, the processor 110 may perform the method/operation by performing one or more instructions constituting the computer program 151. The memory 120 may be implemented as a volatile memory such as RAM, but the technical scope of the present disclosure is not limited thereto.

The bus 130 provides a communication function between the components of the first server 100. The bus 130 may be implemented as various types of buses such as an address bus, a data bus, and a control bus.

The communication interface 140 supports wired/wireless Internet communication of the first server 100. In addition, the communication interface 140 may support various communication methods other than the Internet communication. For this supporting, the communication interface 140 may be configured to include a communication module well known in the technical field of the present invention. In some embodiments, the communication interface 140 may be omitted.

The storage 150 may non-temporarily store the computer program 151. In the case of performing a brain stimulation simulation process according to the preset guide system using the external server through the first server 100, the storage 150 can store various types of information required to provide the brain stimulation simulation process according to the preset guide system using the external server.

The storage 150 may be configured to include a non-volatile memory such as a read only memory (ROM), an erasable programmable ROM (EPROM), an electrically erasable programmable ROM (EEPROM), or a flash memory, a hard disk, a removable disk, a computer-readable recording medium of an arbitrary form well-known in the technical field to which the present invention belongs.

The computer program 151 may include one or more instructions that, when the computer program is loaded on the memory 120, the processor 110 is allowed to perform a method/operation according to various embodiments of the present invention. That is, the processor 110 may perform the method/operation according to various embodiments of the present invention by executing the one or more instructions.

In one embodiment, the computer program 151 may contain one or more instructions to perform the brain stimulation simulation method according to the preset guide system using the external server including a step of allowing the first server to generate the global matrix for performing the brain stimulation simulation on the plurality of objects by using the plurality of brain models for each of the plurality of objects and a step of allowing the second server to be provided with the global matrix generated from the first server and to perform the brain stimulation simulation on the plurality of objects by using the provided global matrix.

The steps of the method or algorithm described in relation to the embodiment of the present invention may be implemented directly in hardware, implemented as a software module performed by hardware, or implemented by a combination thereof. The software module may reside in a random access memory (RAM), a read only memory (ROM), an erasable programmable ROM (EPROM), an electrically erasable programmable ROM (EEPROM), a flash memory, a hard disk, a detachable disk, a CD-ROM, or an arbitrary type of a computer-readable recording medium well known in the art to which the present invention belongs.

The components of the present invention may be implemented as a program (or application) to be performed in combination with a computer that is hardware and stored in a medium. The components of the present invention may be implemented as software programming or software components, and similarly, in the embodiments, various algorithms implemented as data structures, processes, routines, or combinations of other programming components are included, and the algorithms can be implemented in a programming or scripting language such as C, C++, Java, or assembler. Functional aspects can be implemented as algorithms performed by one or more processors. Hereinafter, the brain stimulation simulation method according to the preset guide system using the external server performed by the first server 100 will be described with reference to FIGS. 3 to 10.

FIG. 3 is a flowchart illustrating the brain stimulation simulation method according to the preset guide system using the external server according to another embodiment of the present invention.

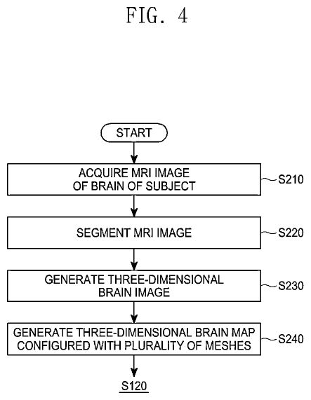

Referring to FIG. 3, in step S110, the first server 100 may generate the brain model for each of the plurality of objects in order to perform the brain stimulation simulation on the plurality of objects. Hereinafter, a process for generating the brain model for each of the plurality of objects performed by the first server 100 will be described in more detail with reference to FIG. 4.

FIG. 4 is a flowchart illustrating a method of generating the three-dimensional brain map by using the brain image of the object in various embodiments.

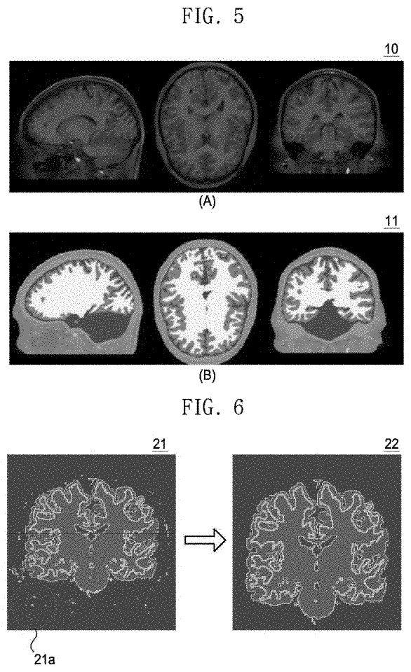

Referring to FIG. 4, in step S210, the first server 100 may acquire a magnetic resonance imaging (MRI) image (for example, 10 in FIG. 5A) of the brain of the object (or the plurality of objects).

Herein, the MRI image of the brain of the object may mean an MRI image captured by photographing the head including the brain of the object. That is, the MRI image of the brain of the object may include not only the brain of the object but also the skull and scalp of the object. For example, the first server 100 may be connected to a computer that is a workstation connected to an MRI image acquisition device and may directly acquire the MRI image of the brain of the object from the MRI image acquisition device through the computer. However, the present invention is not limited thereto.

In step S220, the first server 100 may segment (partition) the MRI image acquired in step S210 into a plurality of areas (for example, 11 in FIG. 5B).

In various embodiments, the first server 100 may generate a plurality of area by analyzing the acquired MRI image and segmenting the MRI image according to region of the brain. For example, the server 100 may segment the MRI image into a white matter area, a gray matter area, a brain spinal fluid area, a skull area, and a scalp area of the brain, but the segmentation is not limited thereto.

In various embodiments, the first server 100 may segment the MRI image into a plurality of areas by analyzing the MRI image with the previously-trained AI model.

Herein, the pre-trained AI model includes one or more batch normalization layers, activation layers, and convolution layers, and the pre-trained AI model may be an AI model learned according to a machine learning-based learning method by setting an MRI image segmented into a plurality of areas according to regions of the brain as learning data (for example, a model learned by using machine learning, particularly, a model learned by using deep learning).

In addition, the pre-trained AI model may be configured to include a horizontal pipeline configured with a plurality of blocks for extracting high-level properties from low-level properties of the MRI image and a vertical pipeline for performing the segmentation by collecting the properties extracted from the horizontal pipelines so as to perform the segmentation on the MRI image with poor image quality, but the AI model is not limited thereto.

In various embodiments, the first server 100 may perform post-processing on the MRI image segmented into the plurality of areas according to the above method.

First, referring to FIG. 6, the first server 100 may perform connected component-based noise rejection on the MRI image segmented into the plurality of areas.

Herein, the connected component-based noise rejection can be utilized in the process of improving the results of MRI image segmentation performed by using a convolutional neural network (CNN). For example, the first server 100 rejects the remaining components 21a from the MRI image 21 segmented into a plurality of areas as illustrated in FIG. 6 except for the connection component, which is the largest chunk, so as to generate the MRI image 22 from which noise is rejected.

Herein, various techniques are known in relation to the method for performing the connection component-based noise rejection, and these various known techniques can be selectively applied depending on the situation. In the present specification, the connection component-based noise rejection method performed by the first server 100 is not disclosed in detail.

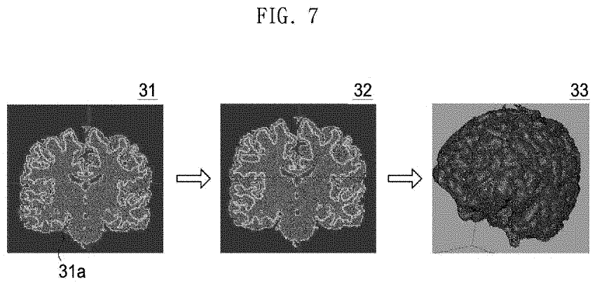

Thereafter, referring to FIG. 7, the first server 100 may perform the hole rejection on the MRI image segmented into the plurality of areas. Herein, the hole rejection can be used to reject a hole, which is one of the errors in convolutional neural network-based segmentation. For example, the first server 100 may generate the MRI image 32 from which the holes are rejected by rejecting at least a portion of the holes 31a included in the MRI image 31 segmented into the plurality of areas.

Herein, with respect to the method for performing the hole rejection, various techniques are known as in the method for performing the connection component-based noise rejection, and these various known techniques can be selectively applied depending on the situation. Therefore, in the present specification, the method of allowing the first server 100 to perform the hole rejection is not disclosed in detail.

In step S230, the first server 100 may generate the three-dimensional brain image (for example, 33 in FIG. 7) by using the MRI image (for example, an MRI image from which noise and holes are rejected) segmented into the plurality of areas.

In step S240, the first server 100 can generate a three-dimensional brain map configured with a plurality of grids (meshes) capable of simulating a transmission procedure of the electric stimulation based on the attributes of each of the plurality of areas included in the three-dimensional brain image generated through step S230. For example, the first server 100 may generate the three-dimensional stereoscopic image configured with a plurality of spatial meshes (volumetric meshes) including a tetrahedron or a hexahedron, or may generate the three-dimensional stereoscopic image configured with a plurality of surface grids (surface meshes) including a triangle or a square. But the present invention is not limited thereto, and the type of grid constituting the three-dimensional stereoscopic image may be set differently depending on the application of simulation.

In various embodiments, the first server 100 may allocate a physical property to each of the plurality of areas included in the three-dimensional brain map. At this time, the first server 100 may determine a type of physical property to be allocated to each of the plurality of areas according to the type of brain stimulation to be simulated.

For example, in the case of simulating the electrical stimulation to the brain with respect to the plurality of objects, the first server 100 may allocate conductivity per tissue to each of the plurality of areas as physical properties. However, the present invention is not limited thereto.

In addition, when ultrasonic brain stimulation for a plurality of objects is to be simulated, the first server 100 can allocate, a density per tissue for each of the plurality of areas and values using the density thereof, λ (first parameter related to bulk modulus and shear modulus), μ (second parameter or stiffness modulus), η (shear or first viscous modulus), and φ (volume or second viscosity coefficient) as physical properties. However, the present invention is not limited thereto.

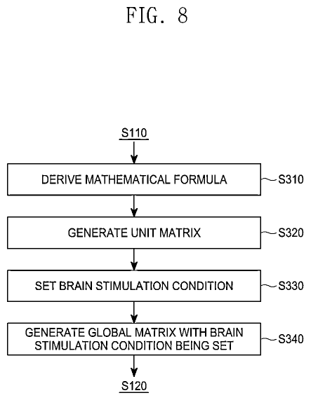

Referring again to FIG. 3, in step S120, the first server 100 can generate the global matrix for performing the brain stimulation simulation on the plurality of objects by using the plurality of brain models for each of the plurality of objects generated through step S110. Hereinafter, a method of generating the global matrix performed by the first server 100 will be described in detail with reference to FIG. 8.

FIG. 8 is a flowchart illustrating the method of generating the global matrix in various embodiments.

Referring to FIG. 8, in step S310, the first server 100 may derive a mathematical formula for performing the brain stimulation simulation.

Herein, the mathematical formula may be a governing equation mathematically describing the relationship between the independent variable and the dependent variable, but the present invention is not limited thereto.

In various embodiments, in order to perform the simulation of applying the electrical stimulation to the brain such as transcranial direct current stimulation (tDCS), the first server 100 may derive the mathematical formula (governing equation) about the distribution of electric potential in the brain generated by applying the electrical stimulation to the brain. For example, the first server 100 may derive the governing equation such as Mathematical Formulas 1 and 2 below by using a quasi-static Maxwell's equation.

$$∇ \cdot ( σ ∇ V ) = 0 in Ω \qquad < Mathematical Formula 1 >$$

$${{σ}_{x} ( \frac{\partial V}{\partial x} )}^{2} + {{σ}_{y} ( \frac{\partial V}{\partial y} )}^{2} + {{σ}_{z} ( \frac{\partial V}{\partial z} )}^{2} = 0 in Ω \qquad < Mathematical Formula 2 >$$

Herein, σₓ may denote the x-axis electrical conductivity (S/m), σ_{y} may denote the y-axis electrical conductivity (S/m), σ_{z} may denote the z-axis electrical conductivity (S/m), V may denote the potential, and Ω may denote an analysis domain (head).

Herein, when it is considered that no current flows out of the analysis domain except for the area to which the electrode is attached, the boundary condition (Neumann boundary condition) as illustrated in Mathematical Formula 3 below may be obtained.

n·(−σ≡V)=0 on Ω <Mathematical Formula 3>

In various embodiments, the first server 100 derives a mathematical formula (governing equation) for performing the brain stimulation simulation, but the first server 100 can determine the form of the derived mathematical formula according to the purpose of performing the brain stimulation simulation.

For example, when the purpose of performing the brain stimulation simulation is time-series current prediction, the first server 100 can derive a mathematical formula based on Maxwell's equation.

In addition, when the purpose of performing the brain stimulation simulation is constant current and low-frequency current prediction, the first server 100 can derive a mathematical formula based on the quasi-static Maxwell's equation as described above.

In addition, when the purpose of performing the brain stimulation simulation is vibration prediction for ultrasound stimulation, the first server 100 can derive a mathematical formula based on linear acoustics. However, the present invention is not limited thereto.

In step S320, the first server 100 may generate a plurality of unit matrices by using the mathematical formula (governing equation) derived in step S310 and may generate the global matrix by using the plurality of unit matrices.

In various embodiments, the first server 100 may generate a stiffness matrix by solving the mathematical formula (governing equation) derived in step S310 by using the Galerkin method and may generate the global matrix by using the generated stiffness matrix.

Herein, the Galerkin method is a method of solving the governing equation by approximating the solution of the governing equation, and the Galerkin method denotes a method of calculating the solution of the governing equation by allowing the weighted average of the residuals to be 0 for residuals (or errors) generated by assuming an approximate solution (for example, a linear combination of test functions (or trial functions)) and substituting the solution into the governing equation.

At this time, in the case of the three-dimensional brain map including a plurality of nodes, since the shape is somewhat complicated, there is a very difficult problem in calculation of the solution of the governing equation that satisfies boundary conditions for all nodes included on the three-dimensional brain map.

In consideration of this problem, the first server 100 can divide the entire analysis domains which are difficult to apply boundary conditions into finite elements which are sub-domains with simple shapes and can calculate the solution of the governing equation by applying boundary conditions to each sub-domain.

More specifically, the first server 100 can group the plurality of nodes included in the three-dimensional brain map into a plurality of groups and can generate the unit matrix for each of the plurality of groups by using the mathematical formula (governing equation) derived in step S310.

For example, the three-dimensional brain map may include a plurality of spatial grids including a tetrahedron, and in the first server 100, a plurality of groups may be generated by grouping four nodes forming a tetrahedral shape, and a stiffness matrix for the four nodes forming the tetrahedral shape may be generated by using the finite element method (FEM).

That is, the first server 100 can set each of the plurality of groups generated by grouping the four nodes forming a tetrahedral shape and segmenting the three-dimensional brain map, which is the entire analysis domain having a somewhat complicated shape, as an individual sub-domain and can generate the stiffness matrix for each of the sub-domains based on the Galerkin method. Hereinafter, the method for the first server 100 to generate the stiffness matrix for each of the plurality of sub-domains and the method for generating the global matrix by using the above method will be described.

First, the first server 100 may define the residual r of each sub-domain (a combination of four nodes forming a tetrahedron) as in Mathematical Formulas 4 to 6 below.

$$r = \frac{\partial }{\partial x} ( {σ}_{x} \frac{\partial V}{\partial x} ) + \frac{\partial }{\partial y} ( {σ}_{y} \frac{\partial V}{\partial y} ) + \frac{\partial }{\partial z} ( {σ}_{z} \frac{\partial V}{\partial z} ) in Ω \qquad < Mathematical Formula 4 >$$

$${R}_{i}^{e} = \int \int {\int }_{{V}^{e}} {N}_{i}^{e} rdV , i = 1 , 2 , 3 , 4 \qquad < Mathematical Formula 5 >$$

$${R}_{i}^{e} = \int \int {\int }_{{V}^{e}} {N}_{i}^{e} [ \frac{\partial }{\partial x} ( {σ}_{x} \frac{\partial {V}^{e}}{\partial x} ) + \frac{\partial }{\partial y} ( {σ}_{y} \frac{\partial {V}^{e}}{\partial y} ) + \frac{\partial }{\partial z} ( {σ}_{z} \frac{\partial {V}^{e}}{\partial z} ) ] dV , i = 1 , 2 , 3 , 4 \qquad < Mathematical Formula 6 >$$

Herein, i may denote a trial function of the n-th node, e may denote a sub-domain (a combination of four nodes forming a tetrahedron), and Rᵢ^{e} may denote a stiffness matrix of the sub-domain.

Thereafter, the first server 100 may derive Mathematical Formula 7 later by performing a chain rule with respect to Mathematical Formula 6 above.

$$\text{?} = \int \int {\int }_{{V}^{e}} \{ \frac{\partial }{\partial x} [ {N}_{i}^{e} ( {σ}_{x} \frac{\partial {V}^{e}}{\partial x} ) ] + \frac{\partial }{\partial y} [ {N}_{i}^{e} ( {σ}_{y} \frac{\partial {V}^{e}}{\partial y} ) ] + \frac{\partial }{\partial z} [ {N}_{i}^{e} ( {σ}_{z} \frac{\partial {V}^{e}}{\partial z} ) ] \} - \{ {σ}_{x} \frac{\partial {N}_{i}^{e}}{\partial x} \frac{\partial {V}^{e}}{\partial x} + {σ}_{y} \frac{\partial {N}_{i}^{e}}{\partial y} \frac{\partial {V}^{e}}{\partial y} + {σ}_{z} \frac{\partial {N}_{i}^{e}}{\partial z} \frac{\partial {V}^{e}}{\partial z} \} dV , i = 1 , 2 , 3 , 4 , \qquad < Mathematical Formula 7 >$$

$$\text{?} \text{indicates text missing or illegible when filed}$$

In addition, the first server 100 may derive the following Mathematical Formula 8 later by applying the divergence theorem to Mathematical Formula 7 above.

$$\text{?} = {∯}_{\text{?}} ( {N}_{i}^{e} ( {σ}_{x} \frac{\partial {V}^{e}}{\partial x} ) + {N}_{i}^{e} ( {σ}_{y} \frac{\partial {V}^{e}}{\partial y} ) + {N}_{i}^{e} ( {σ}_{z} \frac{\partial {V}^{e}}{\partial z} ) ) \cdot \text{?} dS - \int \int {\int }_{{V}^{e}} {σ}_{x} \frac{\partial {N}_{i}^{e}}{\partial x} \frac{\partial {V}^{e}}{\partial x} + {σ}_{y} \frac{\partial {N}_{i}^{e}}{\partial y} \frac{\partial {V}^{e}}{\partial y} + {σ}_{z} \frac{\partial {N}_{i}^{e}}{\partial z} \frac{\partial {V}^{e}}{\partial z} dV , i = 1 , 2 , 3 , 4 \qquad < Mathematical Formula 8 >$$

$$\text{?} \text{indicates text missing or illegible when filed}$$

In addition, the first server 100 may derive Mathematical Formula 9 later by arranging Mathematical Formula 8 above by using the relationship between V^{e} and Vⱼ^{e}.

$${R}_{i}^{e} = {∯}_{\text{?}} ( {N}_{i}^{e} ( {σ}_{x} \frac{\partial {V}^{e}}{\partial x} ) + {N}_{i}^{e} ( {σ}_{y} \frac{\partial {V}^{e}}{\partial y} ) + {N}_{i}^{e} ( {σ}_{z} \frac{\partial {V}^{e}}{\partial z} ) ) \cdot \text{?} dS - {\sum }_{\text{?} = 1}^{\text{?}} {V}_{j}^{e} \int \int {\int }_{{V}^{e}} {σ}_{x} \frac{\partial {N}_{i}^{e}}{\partial x} \frac{\partial {N}_{j}^{e}}{\partial x} + {σ}_{y} \frac{\partial {N}_{i}^{e}}{\partial y} \frac{\partial {N}_{j}^{e}}{\partial y} + {σ}_{z} \frac{\partial {N}_{i}^{e}}{\partial z} \frac{\partial {N}_{j}^{e}}{\partial z} dV , i = 1 , 2 , 3 , 4 \qquad < Mathematical Formula 9 >$$

$$\text{?} \text{indicates text missing or illegible when filed}$$

At this time, in consideration that there is no current source in the brain and the current does not go out of the analysis domain in the area other than the area where the electrode is attached, the first server 100 can set a surface integral part to 0 and derive Mathematical Formula 10 later depending on this.

$${R}_{i}^{e} = {\sum }_{\text{?} = 1}^{\text{?}} {V}_{j}^{e} \int \int {\int }_{{V}^{e}} {σ}_{x} \frac{\partial {N}_{i}^{e}}{\partial x} \frac{\partial {N}_{j}^{e}}{\partial x} - {σ}_{y} \frac{\partial {N}_{i}^{e}}{\partial y} \frac{\partial {N}_{j}^{e}}{\partial y} + {σ}_{z} \frac{\partial {N}_{i}^{e}}{\partial z} \frac{\partial {N}_{j}^{e}}{\partial z} dV , i = 1 , 2 , 3 , 4 \qquad < Mathematical Formula 10 >$$

$$\text{?} \text{indicates text missing or illegible when filed}$$

Herein, Rᵢ^{e} is a stiffness matrix for four nodes forming a tetrahedron, that is, sub-domains and has an i*j matrix (4*4 matrix).

That is, the first server 100 may generate the stiffness matrix for each of the plurality of groups by applying Mathematical Formula 10 to each of the plurality of groups (sub-domains).

Thereafter, the first server 100 may generate the global matrix in a form of a k*k matrix by combining the stiffness matrices for the plurality of groups.

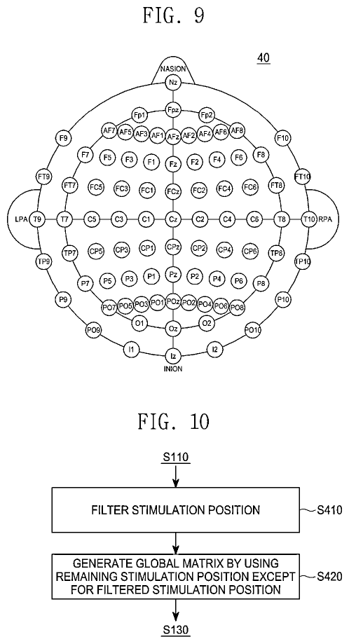

In step S330, the first server 100 may set boundary conditions for performing the brain stimulation simulation by using the global matrix. For example, the first server 100 may be set with a stimulation condition including at least one of a plurality of stimulation positions according to a preset guide system (for example, a 10-20 SYSTEM, 40 in FIG. 9), the number of electrodes attachable to the plurality of stimulation positions and the intensity of brain stimulation.

In step S340, the first server 100 generates a global matrix assembly with boundary condition including the brain stimulation conditions by allocating the stimulation conditions set in step S330 to the global matrix generated in step S320.

In various embodiments, the first server 100 may generate a brain stimulation condition list by listing the brain stimulation conditions set through step S340. The first server 100 matches and combines the list of brain stimulation conditions with the global matrix assembly and provides them to the second server 200, so that the second server 200 can perform brain stimulation simulations according to the brain stimulation conditions.

In various embodiments, the first server 100 can filter the stimulation positions corresponding to the preset condition among the plurality of stimulation positions according to the preset guide system and can generate the global matrix so as to perform the brain stimulation simulation on the object by using the remaining stimulation positions excluding the filtered stimulation positions among the plurality of stimulation positions. Hereinafter, the embodiment will be described with reference to FIGS. 10 to 13.

FIG. 10 is a flowchart illustrating the method for performing the brain stimulation simulation by filtering the stimulation positions in various embodiments.

Referring to FIG. 10, in step S410, the first server 100 may filter the stimulation positions corresponding to the preset conditions among the plurality of stimulation positions according to the preset guide system.

In various embodiments, the first server 100 may filter at least one stimulation position by using the head image for the object.

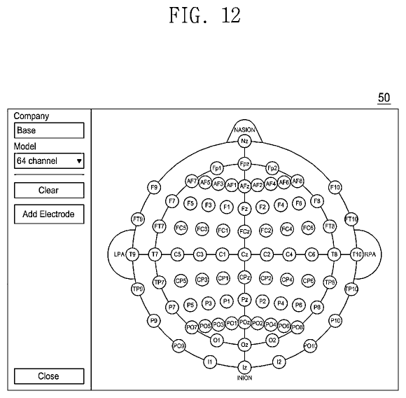

First, the first server 100 may acquire the head image generated by photographing the head of the object and may set one or more reference stimulation positions based on the acquired head image. For example, the first server 100 may provide a UI (for example, 50 in FIG. 12) to the user terminal 300, may output the plurality of stimulation positions according to the preset guide system through the UI, and may set the reference stimulation positions by selecting one or more stimulation positions among the output plurality of stimulation positions as the reference stimulation positions. However, the present invention is not limited thereto, and various methods such as a method of automatically setting a reference stimulation position for calculating the plurality of stimulation positions according to the preset guide system by performing an image analysis of the head image of the object may be applied.

Thereafter, the first server 100 may set the plurality of stimulation positions based on one or more reference stimulation positions. For example, in the case where the total number of reference stimulation positions set according to the above method is four and the four stimulation positions corresponding to the nasion, the inion, the left ear, and the right ear of each object are Nz, Iz, LPA, and RPA, the first server 100 can calculate the point where the first connecting line that connects the stimulation positions Nz and Iz and the second connecting line that connects the stimulation positions LPA and RPA intersects as the central coordinate and can derive the coordinate system for the plurality of stimulation positions according to the 10-20 system using the distance information on the first and second connecting lines based on the central coordinate. As an example, the first server 100 may derive the coordinate system of the 10-20 system so as to have a position where the first connection line and the second connection line are segmented with distances of 10% or 20%, respectively, based on the central coordinate.

Thereafter, the first server 100 may set a filtering target area (for example, an area serving as a reference for filtering the stimulation position) by using the plurality of stimulation positions set on the head image and may filter at least one stimulation positions based on the set filtering target area.

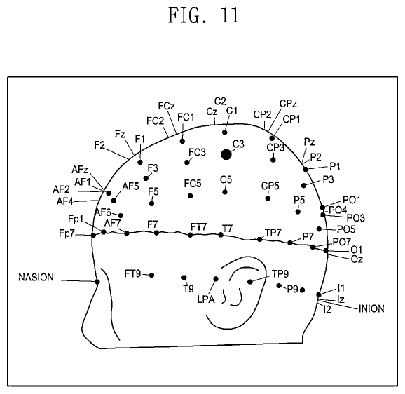

In various embodiments, the first server 100 may set a plane including one or more reference stimulation positions as the filtering target area and may filter at least one stimulation position located on the plane set as the filtering target area based on the plane set as the filtering target area. For example, as illustrated in FIG. 11, in the case where one or more reference stimulation positions are the four stimulation positions Nz, Iz, LPA, and RPA corresponding to the nasion, the inion, the left ear, and the right ear, the first server 100 can set the plane including Nz, Iz, LPA, and RPA as the filtering target area and can filter all the stimulation positions located on the plane including Nz, Iz, LPA, and RPA.

In various embodiments, the first server 100 may filter all the stimulation positions located at the bottom of the corresponding plane based on the plane set as the filtering target area. For example, when one or more reference stimulation positions set by the user are Fpz, T7, Oz, and T10, the first server 100 can filter the stimulation positions Nz, Iz, LPA, and RPA located at the bottom of the plane including Fpz, T7, Oz, and T10.

That is, due to the shape of the head or the ear, it is difficult to attach the electrode to the stimulation positions corresponding to the nasal muscles, the laryngeal pole, the left ear, and the right ear, or it is difficult to attach the electrode to the correct position even if the electrode is attached. Therefore, the stimulation positions corresponding to these positions need to be filtered.

In various embodiments, the first server 100 can detect an area in which the electrodes cannot be attached on the head of the object by analyzing the head image, can set the detected area in which the electrodes cannot be attached as a filtering target area, and can filter at least one stimulation position included on the filtering target area. For example, when there is an area with a metallic substance (clip, coil, metabolic foreign body, or the like) on the brain of the object or when there is an injury such as a scalp disease or wound, there is a problem in that it is difficult to attach the electrodes to the area and apply the electrical stimulation. In consideration of this problem, the first server 100 can detect the area in which the electrodes cannot be attached as described above by analyzing the head image of the object through image analysis and can filter the stimulation positions included in the detected area.

In step S420, the first server 100 may generate the global matrix by using only the stimulation positions remaining except for the stimulation positions filtered through step S410. For example, the first server 100 can generate the global matrix except for the filtered stimulation position by combining only the stiffness matrices generated corresponding to the remaining sub-domains except for the stiffness matrix generated corresponding to the sub-domain corresponding to the stimulation position filtered according to the above method among the plurality of sub-domains (a plurality of groups).

Referring again to FIG. 3, in step S130, the first server 100 may be connected to the second server 200 through the network 400 and may provide the global matrix (the global matrix assembly with boundary condition including stimulation conditions) generated according to the above method to the second server 200.

In step S140, the second server 200 may perform the brain stimulation simulation by using the global matrix provided from the first server 100 through step S130.

First, the second server 200 can derive a mathematical formula for the brain stimulation simulation by using the global matrix. For example, when the brain stimulation to be simulated is the electrical stimulation, the second server 200 can derive the linear equation (for example, AV=R, where R is the residual) for the electrical stimulation simulation and may finally derive the Mathematical Formula 11 later by allocating 0 to R so that the residual becomes 0 and adding the brain stimulation condition to the linear equation.

AV=b <Mathematical Formula 11>

Herein, A may denote the global matrix (k*k matrix), V may denote the electric potential value (k*1 matrix, vector) generated by applying the electrical stimulation to the brain, and b may denote the force vector (k*1 matrix, force vector).

Thereafter, the second server 200 may perform the brain stimulation simulation based on the list of the brain stimulation conditions matched with the global matrix and calculate the electric potential value V that satisfies the above linear equation as a result of the brain stimulation simulation.

In various embodiments, the second server 200 may calculate the electric potential value V that satisfies the linear equation according to Mathematical Formula 11 by using at least one among the conjugate gradient method and the double -conjugate gradient method. However, the present invention is not limited thereto, and various methods for calculating a solution of a linear equation may be applied.

In various embodiments, the second server 200 can convert the electric potential value calculated by using at least one of a conjugate gradient method and double conjugate gradient method into the electric field value. For example, since a relationship between the electric field and the electric potential is established as shown in Mathematical Formula 12, the second server 200 can convert the electric potential value V into the electric field value E by using Mathematical Formula 12.

E=−∇V <Mathematical Formula 12>

In step S150, the second server 200 can provide the result (for example, the electric field value (k*1 matrix, vector) converted from the calculated electric potential value) of the brain stimulation simulation generated by performing the brain stimulation simulation based on the global matrix in the above manner to the first server 100 through the network 400.

In step S160, the first server 100 may collect the results of the brain stimulation simulation provided from the second server 200.

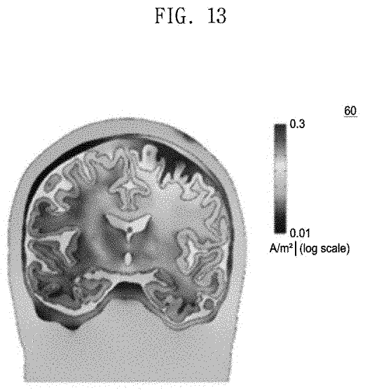

In various embodiments, the first server 100 may generate the final result of the brain stimulation simulation by matching the result of the brain stimulation simulation provided from the second server 200 on the three-dimensional brain map. For example, as illustrated in FIG. 13, the first server 100 may generate the final result of brain stimulation simulation by converting the electric field value corresponding to a specific position into a preset color (a preset color according to the size and range of the electric field value) and displaying the preset color on the three-dimensional brain map based on the result of the brain stimulation simulation provided from the second server 200.

In step S170, the first server 100 may be connected to the user terminal 300 through the network 400, and by providing the final result of the brain stimulation simulation (for example, 60 in FIG. 13) to the user terminal 300, the display of the user terminal 300 can be allowed to output the final result of the brain stimulation simulation.

That is, as described above, since various information and data (for example, the brain stimulation condition list, the governing equation, the boundary conditions, the global matrix, or the like) provided from the first server 100 to the second server 200 is non-identified information that does not include information (for example, outside information of the patient) that can infer a specific object and can only be identified through the three-dimensional brain map that exists in a medical institution and the second server 200 performs the brain stimulation simulation without information by which the specific object can be inferred, there is an advantage in that personal information about the object can be prevented from being leaked to the outside.

The above-described brain stimulation simulation method according to the preset guide system using the external server is described with reference to the flowchart illustrated in the drawings. For a simple description, the brain stimulation simulation method according to the preset guide system using the external server is described with a series of blocks, but the present invention is not limited to the order of the blocks. Some blocks may be performed in order different from the order illustrated and described in this specification or may be performed simultaneously. In addition, new blocks not described in the present specification and drawings may be added, or some blocks may be deleted or changed.

In addition, although it is described that the above-described brain stimulation simulation method according to the preset guide system using the external server, generates the global matrix that is an anonymized data by using medical data, transmits the global matrix to the external server, and processes a task (for example, the brain stimulation simulation) which is difficult to process through an internal server in a medical institution through the external server with relatively high performance in comparison to the internal server, the present invention is not limited thereto, as performing physical analysis of medical images by analyzing medical images through the external server, the brain stimulation simulation method can be applied to all fields that anonymize internal data that requires anonymization and transmit the internal data to the external server, and process the anonymized internal data through the external server.

In the above, embodiments of the present invention have been described with reference to the accompanying drawings, but the ordinarily skilled in the art to which the present invention belongs can understand that the present invention can be carried out in other specific forms without changing technical spirit or essential features. Therefore, it should be understood that the embodiments described above are illustrative in all respects and are not restrictive.

1. A brain stimulation simulation method according to a preset guide system using an external server, the method performed by a computing device, the method comprising:
a first server generating a global matrix for performing brain stimulation simulation on a plurality of objects by using a plurality of brain models for each of the plurality of objects; and
a second server being provided with the generated global matrix from the first server and performing the brain stimulation simulation on the plurality of objects by using the provided global matrix.

2. The method according to claim 1, wherein the generating the global matrix includes:
acquiring an MRI image of the plurality of objects;
segmenting the acquired MRI image into a plurality of areas;
generating a three-dimensional brain image by using the MRI image segmented into the plurality of areas;
generating a three-dimensional brain map configured with a plurality of meshes based on properties of each of the plurality of areas included in the generated three-dimensional brain image; and
generating the global matrix by using the generated three-dimensional brain map.

3. The method according to claim 2, wherein the generating the global matrix by using the generated three-dimensional brain map includes:
deriving a mathematical formula for performing the brain stimulation simulation;
grouping a plurality of nodes included in the generated three-dimensional brain map into a plurality of groups and generating a unit matrix for each of the plurality of groups by using the derived mathematical formula; and
generating one global matrix by combining the generated unit matrix.

4. The method according to claim 3, wherein the deriving the mathematical formula includes deriving the mathematical formula for performing the brain stimulation simulation, a form of the derived mathematical formula being determined according to a purpose of performing the brain stimulation simulation, wherein the purpose includes at least one among time-series current prediction, constant current and low-frequency current prediction, and vibration prediction for ultrasound stimulation.

5. The method according to claim 3,
wherein each of the plurality of groups includes four nodes forming a tetrahedral shape, and
wherein the generating the unit matrix for each of the plurality of groups includes generating a stiffness matrix for the four nodes forming the tetrahedral shape by using a finite element method (FEM).

6. The method according to claim 2, wherein the segmenting into the plurality of area includes allocating a physical property for each of the plurality of areas to each of the plurality of areas generated by segmenting the acquired MRI image, a type of the physical property allocated to each of the plurality of areas being determined according to a type of brain stimulation to be simulated.

7. The method according to claim 2, wherein the generating the global matrix further includes setting a brain stimulation condition for performing the brain stimulation simulation, the set brain stimulation condition including at least one of a plurality of stimulation positions according to the preset guide system, a number of electrodes attachable to the plurality of stimulation positions, and an intensity of brain stimulation.

8. The method according to claim 2, further comprising the first server being provided with a result of brain stimulation simulation for the plurality of objects from the second server and outputting a combination of the provided result of the brain stimulation simulation and the generated three-dimensional brain map.

9. The method according to claim 1, wherein the performing the brain stimulation simulation includes:
deriving a linear equation for the provided global matrix by using the provided global matrix; and
calculating a solution of the derived linear equation as the result of the brain stimulation simulation.

10. A brain stimulation simulation system according to a preset guide system using an external server, comprising:
a first server generating a global matrix for performing brain stimulation simulation on a plurality of objects by using a plurality of brain models for each of the plurality of objects; and
a second server being provided with the generated global matrix from the first server and performing the brain stimulation simulation on the plurality of objects by using the provided global matrix.
